(19) United States
(12) Patent Application Publication
(10) Pub. No.: US 2014/0207188 A1
(43) Pub. Date: Jul. 24, 2014

(54) SURGICAL SUTURE RETRIEVING AND PASSING SYSTEM

(71) Applicant: Howmedica Osteonics Corp., Mahwah, NJ (US)

(72) Inventors: Ryan E. Yearsley, Denver, CO (US); Curtis Custer Edwards, Denver, CO (US)

(73) Assignee: HOWMEDICA OSTEONICS CORP., Mahwah, NJ (US)

(21) Appl. No.: 14/104,480

(22) Filed: Dec. 12, 2013

Related U.S. Application Data
(60) Provisional application No. 61/755,654, filed on Jan. 23, 2013.

(51) Int. Cl. A61B 17/04; A61B 17/06

(52) U.S. Cl. CPC A61B 17/0485; A61B 17/06004
USPC 606/224

(57) ABSTRACT

A suture retaining device, which has an elongate body having a cross section that is at least partially circular, and a head portion joined to the elongate body. The head portion has a sidewall that defines a cavity for receipt of a suture. The sidewall also has an internal surface that is at least partially flat, and the cavity is enclosed by the sidewall. The suture retaining device may be utilized in conjunction with a suture passing device, which has a body that includes a distal end, proximal end and outer surface; an internal passageway extending through the body from the distal end to the proximal end; and a contoured portion defining a recess in the body, the recess extending radially from the outer surface to the internal passageway, the contoured portion is homogenous.

### CROSS-REFERENCE TO RELATED APPLICATION

The present application claims the benefit of the filing date of U.S. Provisional Patent Application No. 61/755,654 filed Jan. 23, 2013, the disclosure of which is hereby incorporated herein by reference.

### FIELD OF THE INVENTION

The present invention relates to surgical suturing instruments, and more particularly to hand manipulated instruments for retrieving and passing a surgical suture through tissue.

### BACKGROUND OF THE INVENTION

Minimally invasive surgery, such as arthroscopic surgery, and open surgery performed in narrow body cavities require surgeons to use surgical instruments as extensions of their hands. Surgeons can use these instruments to internally close wounds, reattach separated tissue, form a new structure with transplanted tissue, provide support structures, among other procedures. Consequently, a number of procedures require a suture to be passed through tissue and retrieved from the exiting side.

Certain instruments exist to provide a surgeon the capability of passing a suture through multiple segments of tissue where it is not feasible or preferable to do so by hand. U.S. Pat. No. 6,077,277, U.S. Pat. No 7,704,262, and U.S. Pat. No. 8,066,718 describe some of these types of instruments. Several of these devices suffer from increased risk of failure due to a number of moving parts. Also, many of these moving parts unnecessarily increase the number of bacteria trapping surfaces, thereby increasing the risk of infection and complicating sterilization. Therefore, these instruments are characteristically employed as single-use devices. Further, many of the mechanisms of the prior art unduly constrain the ability of the surgeon to manipulate the mechanism and receive accurate feedback.

Another aspect of these instruments, which is partially due to their single-use nature, is that they are not precision engineered for optimal configuration. This has led to cannulated needles that are much larger in diameter than instruments used through their cannulated passageways. This can increase trauma to the tissue penetrated by the cannulated needle and can permit the suture retrieving device to move when not desired. Additionally, these cannulated needles can be susceptible to bending, which further dictates that they be disposable, single-use devices.

Therefore, there is a need for a surgical suturing system that has a simple, ergonomic operating mechanism that is reliable, reusable, and precision engineered to provide enhanced operational feedback, situational flexibility, and sterilization maintenance.

### BRIEF SUMMARY OF THE INVENTION

Disclosed herein are instruments for retrieving and passing a surgical suture through tissue. According to one aspect of the present invention, a suture retaining device includes an elongate body that has a cross section that is at least partially circular. The suture retaining device further includes a head portion joined to the elongate body. The head portion has a sidewall defining a cavity for receipt of a suture. The sidewall includes an internal surface that is at least partially flat, and the cavity is enclosed by the sidewall.

In alternate embodiments, the head portion can be collapsible and joined to the elongate body, with the head portion having a sidewall defining a cavity for receipt of a suture, the sidewall having an internal surface that is at least partially flat. The sidewall may form a diamond configuration that is collapsible. The head portion may be made from a memory metal, such as nitinol.

Another aspect of the present invention is a suture retaining device including an elongate body that is at least partially helical. The suture retaining device includes a head portion joined to the elongate body. The head portion has a sidewall that defines a cavity for receipt of a surgical suture. The sidewall has an internal surface that is at least partially flat. In one embodiment, the elongate body may comprise a double helix. In another aspect of the present invention, the elongate body may be configured as a helix and an elongate rod. The helix may be wound about the elongate rod.

A further aspect of the present invention is a suture passing device including a body having a distal end, a proximal end, and an outer surface. The suture passing device further includes an internal passageway extending through the body from the distal end to the proximal end. Also included is a contoured portion that defines a recess in the body. The recess extends radially from the outer surface to the internal passageway. The contoured portion may be homogenous or constructed of uniform composition throughout.

In one embodiment, the contoured portion may include a ridge that is substantially perpendicular to the internal passageway. The ridge may include a depression that is aligned with the internal passageway. The body may be tapered for conformity to a hand grip.

According to another aspect of the present invention, the suture passing device may include a needle having a cannulated passageway in fluid communication with the internal passageway of the handle. The distal end of the body may be configured to connect to a cannulated needle, wherein connecting the distal end of the body to the cannulated needle places the internal passageway in fluid communication with the cannulation of the cannulated needle. The suture passing device may include a suture retaining device that is slidably and rotatably engaged with the internal passageway of the suture passing device.

In one aspect of the present invention, a suture passer device includes a body having a distal end, a proximal end, and an outer surface. The suture passer device also includes an internal passageway extending through the body from the distal end to the proximal end. Additionally, a contoured portion defines a recess in the body. The recess extends radially from the outer surface to the internal passageway. Also included is a needle having a cannulated passageway in fluid communication with the internal passageway of the handle.

A further aspect of the present invention is a suture retaining device including a length of material having two end portions and an intermediate portion between the end portions. Each end portion has a circular cross section and at least a portion of the intermediate portion has a flattened cross section. The cross section of the end portions can be the same or different, and either can be square, triangular, oval, or any other shape instead of circular. The length of material is bent into a configuration in which the intermediate portion forms a head portion having a closed loop and the end portions together form a body.

A further aspect of the present invention is a suture retaining device including a length of material having two end portions and an intermediate portion between the end portions. At least a portion of the intermediate portion has a flattened cross section. The length of material is bent into a configuration in which the intermediate portion forms a head portion having a closed loop and wherein the end portions are wound together to form a body.

In accordance with other embodiments of the aforementioned two aspects, the end portions can be configured in a double helix construction to form the body. The end portion can be configured such that one end portion is substantially linear and the other end portion is substantially helical and wound about the linear end portion. The head portion can be generally formed into a diamond configuration. At least the head portion can be made from a memory metal. The memory metal can be nitinol.

### BRIEF DESCRIPTION OF THE DRAWINGS

FIG. 1A is a perspective view of a portion of an embodiment of a suture shuttle device having a head portion and an elongate body portion.

FIG. 1B is a cross sectional view of the head portion of the suture shuttle device of FIG. 1A taken along line 1B-1B.

FIG. 1C is a cross sectional view of the elongate body portion of the suture shuttle device of FIG. 1A taken along line 1C-1C.

FIG. 2 is a top view of the suture shuttling device shown in FIG. 1A.

FIG. 3 is a perspective view of the entire suture shuttle device shown in FIG. 1A.

FIG. 4 is a perspective view of a suture passer handle.

FIG. 5 is a front view of the suture passer handle shown in FIG. 4.

FIG. 6 is a top view of the suture passer handle shown in FIG. 4.

FIG. 7 is a back view of the suture passer handle shown in FIG. 4.

FIG. 8 is a bottom view of the suture passer handle shown in FIG. 4.

FIG. 9 is a front view of a suture passer needle.

FIG. 10 is a top view of the suture passer needle shown in FIG. 9.

FIG. 11 is a bottom view of the suture passer needle shown in FIG. 9.

FIG. 12A is an enlarged view of a distal end of certain existing instruments.

FIG. 12B is an enlarged view of a distal end of the suture passer needle of FIG. 9

FIG. 12C is a top view of 45 degree right and left configurations of the suture passer needle of FIG. 9.

FIG. 12D is a top view of various sized crescent configurations of the suture passer needle of FIG. 9.

FIG. 12E is a top view of 25 degree right and left configurations of the suture passer needle of FIG. 9.

FIG. 12F is a top view of 30 and 60 degree straight configurations of the suture passer needle of FIG. 9.

FIG. 13 shows an assembly step of a suture passer assembly.

FIG. 14A shows the suture passer assembly of FIG. 13 as assembled and with the suture shuttle of FIG. 1A loaded into the suture passer assembly.

FIG. 14B shows an enlarged view of the suture shuttle extending from the suture passer assembly as shown in FIG. 14A.

### DETAILED DESCRIPTION

As used herein, when referring to the surgical instruments of the present invention, the term “proximal” means closer to the surgeon or in a direction toward the surgeon, and the term “distal” means more distant from the surgeon or in a direction away from the surgeon. The term “lateral” means away from the midline of the surgical instrument of the present invention.

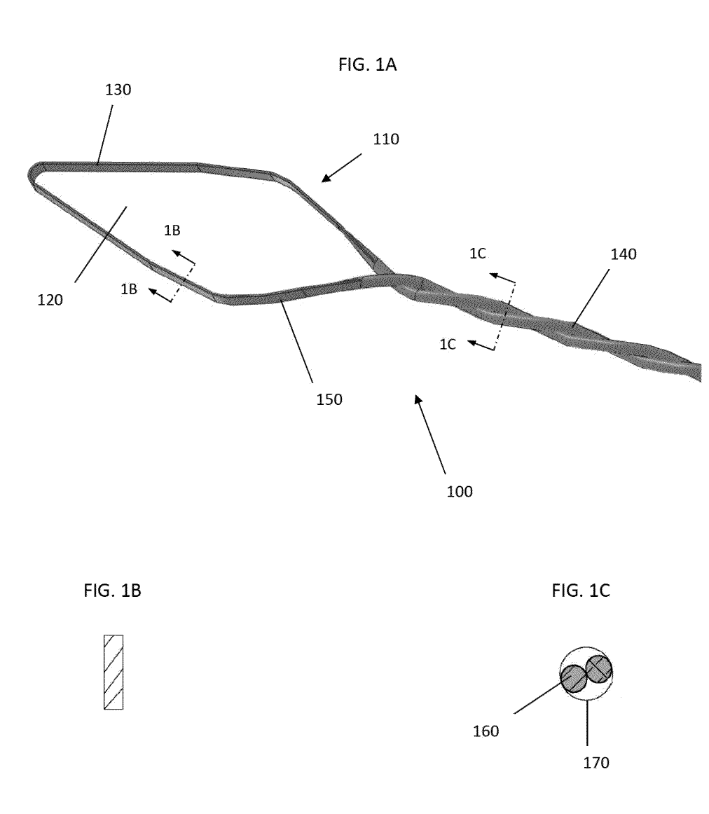

FIGS. 1A-3 show a suture shuttle 100 or suture retaining device in accordance with an embodiment of the present invention. Suture shuttle 100 generally includes a head portion 110 and an elongate body 140. The head portion 110 includes a sidewall 150, which forms a cavity 120 for receiving a suture. The head portion 110 may be diamond shaped, which not only allows the head portion 110 to collapse when introduced to a narrow area, for example a cannula of a needle, but it also promotes movement of a suture to the distal end of the head portion 110 where the head portion is generally the narrowest. This configuration also improves the ability of the collapsed head portion 110 to grip the suture at regions other than the distal end of the head portion 110.

The suture shuttle 100 may be made from any biocompatible material, such as titanium, stainless steel, plastic, ceramic, etc. However, at least the head portion 110 is preferably made from a memory metal, for example nitinol, to aid in the collapsibility and re-expansion of the head portion 110.

The diamond shaped nature of head portion 110 and the fact that shuttle 100 is comprised of a memory metal allows head portion 110 to press outwardly against the inside passageways of the suture passer handle 200 and cannulated needle 300, as described below. This outward pressure provides a frictional force that enhances the ability of shuttle 110 to maintain its position within the passageways of handle 200 and needle 300, even if the device is held in a vertical orientation that may otherwise permit shuttle 100 to slide out of the device.

FIG. 1B shows a cross sectional view of the head portion 110 taken along line 1B-1B of FIG. 1A. Referring to FIGS. 1A and 1B, the head portion 110 preferably has a rectangular cross section with a flat inner surface 130. The combination of a nitinol material with the rectangular cross section improves the collapsibility of the head portion 110 by allowing for the reduction of the thickness of the sidewall 150 without sacrificing structural integrity as might occur with a circular cross section. However, circular cross sections of material may also be used to construct head portion 110 without departing from the present invention.

FIG. 1C shows a cross sectional view of the elongate body 140 having the structure of a double helix. Referring to FIGS. 1A-3, the double helix is shown as two rounded wire-like structures being twisted with respect to each other. As demonstrated by FIG. 1C, the cross section of the double helix has two circular cross sectional components 160 formed by the wire-like structures, respectively, that are in close proximity to each other.

FIG. 1C also illustrates that the wire like structures twisted into an arrangement of a double helix forms a cylindrical diameter 170 along the length of the double helix, wherein the cylindrical diameter 170 includes the diameters of the two circular cross sectional components 160. In one embodiment the two circular components 160 may abut each other such that the cylindrical diameter 170 is equal to the combination of the diameters of the two circular components 160. In other embodiments, there may be space between the two circular components 160 in which the cylindrical diameter 170 would include the diameters of the circular components 160 and the space between them. In additional embodiments, a sheath may be provided around the two circular components 160. This sheath can be shrunk around the wire-like structures to more closely mimic and conform to the outer contour of the helical structure, in which case the cylindrical diameter 170 would include the diameters of the two circular components and the thickness of the sheath. This cylindrical diameter 170 is illustrative of the cylindrical nature of the double helix of the elongate body 140, which facilitates translational and rotational movement of the suture shuttle 100. Further, the helical nature of the elongate body 140 produces optimum contact with the cannulated passageway 340 to provide just enough friction to hold the suture shuttle 100 static but not hinder movement of the suture shuttle 100 when movement is desired.

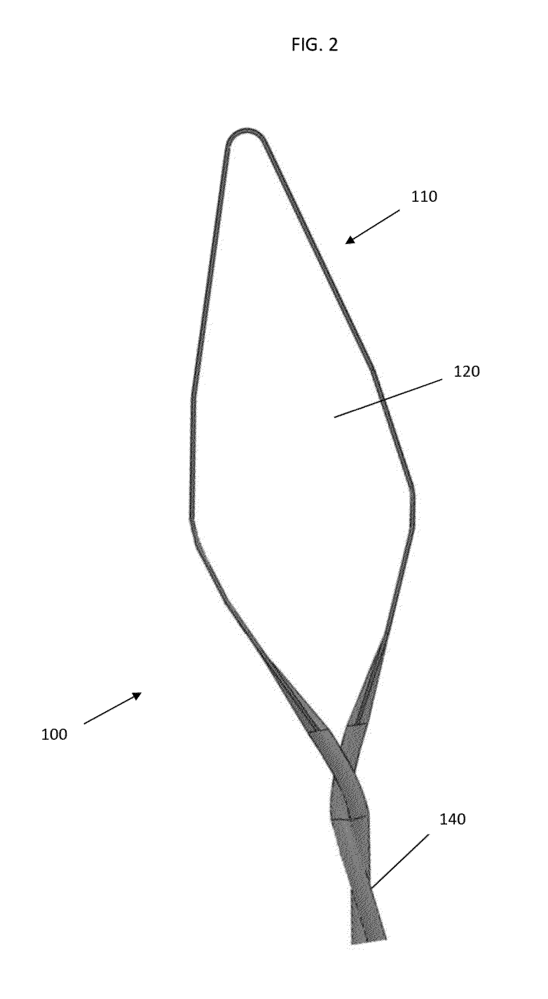

As shown in FIGS. 1A-2, the suture shuttle 100 is constructed from a single, monolithic wire-like structure with a portion of it flattened to form the head portion 110, and the remainder twisted with respect to itself to form the double helix of the elongate body 140. However, the head portion 110 may be provided separately from and attached to body 140 in any number of ways recognized by those skilled in the art, for instance welding or clamping. Body 140 may also be comprised of two separate wound wires, with either of the wires being monolithically connected with the head portion 110, or with the head portion being a third separate element.

Alternatively, the body 140 may also be composed of a single rod with a circular cross section (not shown), either monolithically connected with or separately attached to head portion 110. In this scenario, the head portion may be attached to the single rod in any number of ways recognized by those skilled in the art, for example a portion of the rod can be split, flattened and welded to form the head portion. The cylindrical nature of the elongate body 140 may be realized in the form of a rod of a circular cross section with a single helix wrapped around the rod (not shown). The head portion 110 could be formed in the same manner as the double helix embodiment, with the exception that only one portion of the wire-like structure is twisted with respect to itself.

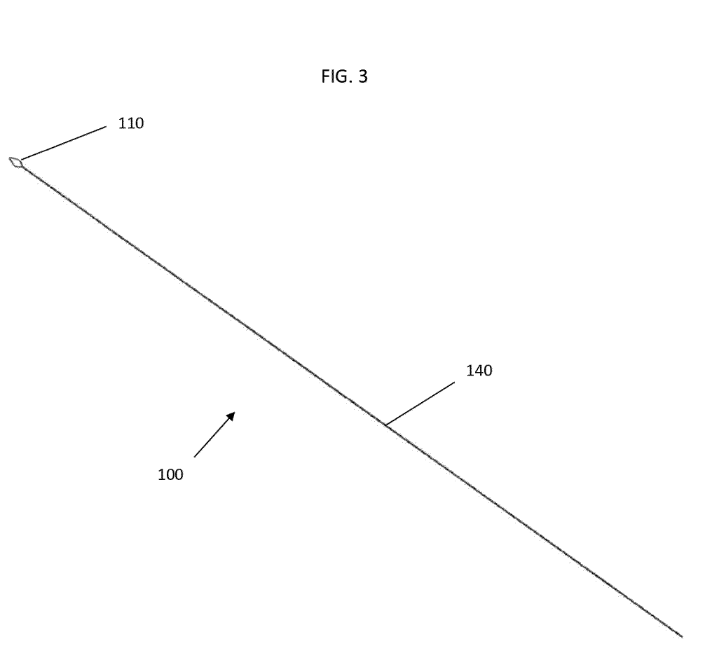

In a preferred embodiment, suture shuttle 100 is manufactured by first providing a single length of material having a circular cross section. The portion of the material that is to form head portion 110 is flattened into a rectangular cross section. The portion of the material that comprises body 140 remains circular in cross section. The rectangular cross section of the material at head portion 110 can be formed by hammering or otherwise flattening the circular cross section of the initially provided material. The length of material can then be bent so that the portion with the flat cross section is shaped into head portion 110, as shown in FIGS. 1 and 2, and the remaining circular cross sectional portions are twisted together to create body 140. A sheath can then be provided over body 140, and shrunk to fit the contour of body if desired. As shown in FIG. 3, body 140 can include a length that is much greater than the length of head portion 110. In certain embodiments, the length of body 140 can be ten or more times the length of body 140.

In alternative embodiments, a suture shuttle in accordance with the present invention can include a head portion 110 at each end of the shuttle. Thus, both of the leading and trailing can be configured to include head portions 110, which can be of the same or different sizes. Additional embodiments can be configured such that the head portion 110 is actually disposed along the length of the body of the shuttle rather than at one end thereof. Multiple head portions 110 can be disposed on shuttle, at either or both ends or only along a middle portion thereof.

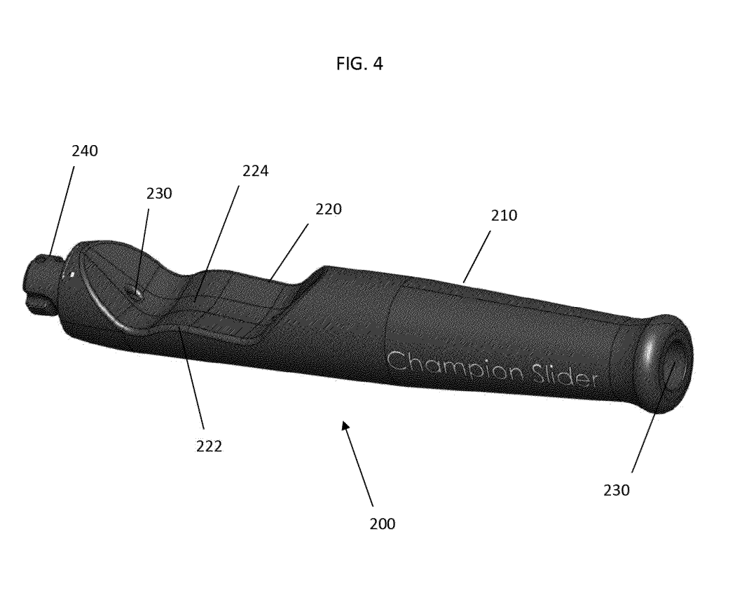

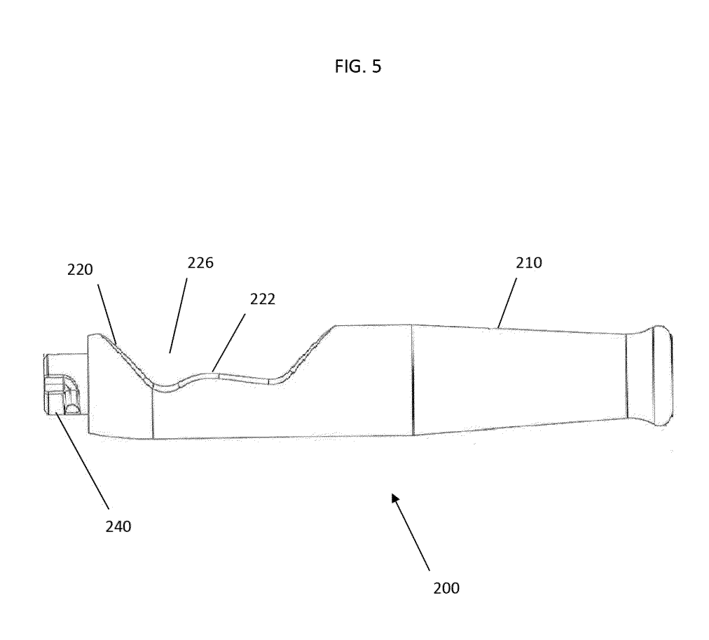

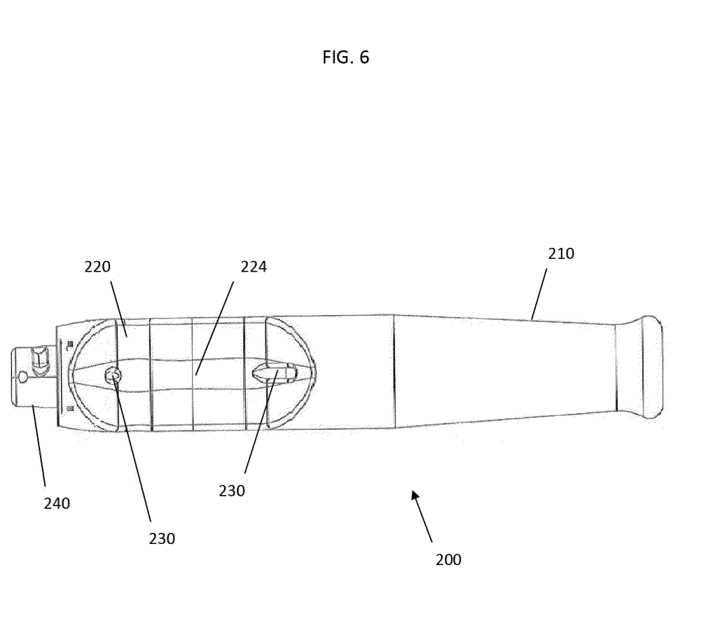

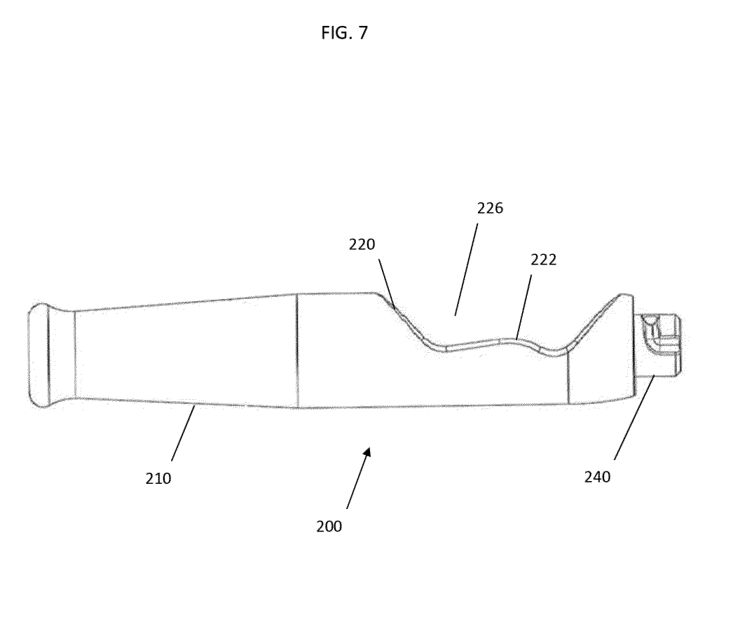

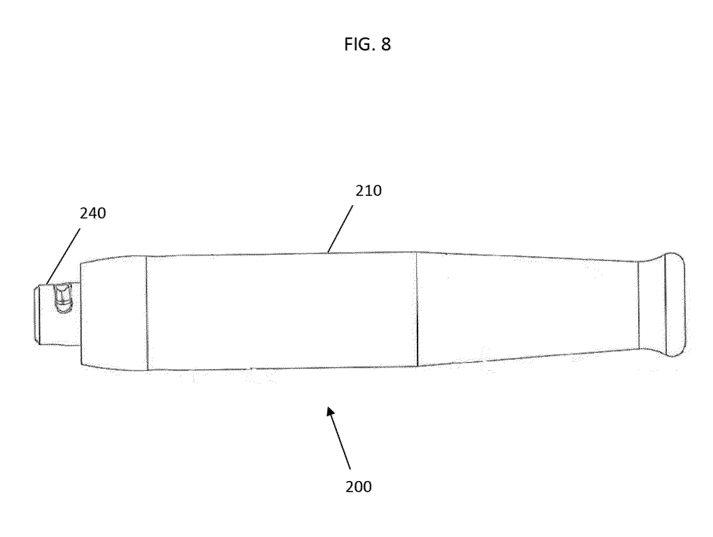

FIGS. 4-8 show a suture passer handle 200 in connection with another embodiment of the present invention. The suture passer handle 200 generally includes a body 210 that defines an internal passageway 230, a contoured portion 220, and a mating portion 240 for engagement with a cannulated needle, described below. The mating portion 240 is a feature that lends the suture passer handle 200 to being reusable, even where a cannulated needle 300, described below, is discarded. While the embodiment demonstrated by FIGS. 4-8 shows a mating portion 220, the suture passer handle may be provided with a cannulated needle integrated into the body 210. The suture passer handle 200 is preferably monolithically constructed, i.e., machined or molded entirely from one piece of material. Thus, the suture passer handle requires no moving parts for its operation, which enhances sterilization, reliability and reusability of the suture passer handle 200. The suture passer handle 200 is preferably made from a biocompatible material, such as polymer, ceramic, and/or metal, such as titanium, for example.

The body 210 may be ergonomically shaped to fit comfortably within a hand of a user, such as having a gradual taper. The internal passageway 230 extends throughout the suture passer handle 200 from the proximal end to the distal end, and is dimensioned to accommodate the elongate body 140 of the suture shuttle 100 described above. In one embodiment, internal passageway 230 is defined by a substantially uniform cylindrical passage provided through handle 200. The portions of handle 200 coinciding with the cylindrical passage define the boundaries of internal passageway 230 and serve to enclose its entire circumference, thereby simplifying loading of suture shuttle 100 into internal passageway 230.

The contoured portion forms a recess 226 in the body 210 near the distal end of the suture passer handle 200. The recess 226 extends radially into the suture passer handle 200 so that the internal passageway 230 is exposed when viewing the contoured portion 220. When a suture shuttle 100 is loaded into the suture passer handle 200 via the internal passageway 230, the recess 226 allows the surgeon to have controllable access to the elongate body 140 of the suture shuttle 100, such that a thumb or finger of the surgeon can be used to contact suture shuttle 100 to pass it through passageway 230. The contoured portion 220 is formed near the distal end so that the surgeon can grip the proximal portion of the body 210 while using his or her thumb to manipulate the suture shuttle 100.

A ridge 222 is provided by contoured portion 220 within recess 226 that runs along the contoured portion 220 in a direction that is generally perpendicular to the internal passageway 230. In this way, ridge 222 can be configured to abut, or even to slightly coincide with or overlap, the substantially uniform cylindrical passage that defines passageway 230. That is, ridge 222 can be configured such that it would abut or overlap an imaginary boundary of the cylindrical passage that would extend between the sections of the passageway 230 located in the proximal and distal aspects of body 210.

In one embodiment, ridge 222 overlaps or intersects the cylindrical passage that defines passageway 230. Such a configuration of ridge 222 allows the generally cylindrically configured elongate body 140 of suture shuttle 100 to rest against ridge when shuttle 100 is disposed within passageway 230 adjacent ridge 222. The slight pressure of ridge 222 against shuttle 100, and the friction imparted on shuttle 100 by ridge 222, aids in maintaining shuttle 100 at a particular location within handle 200. Manipulation of shuttle 100 is even further enhanced by the texture created by the double helix of the elongate body 140 where the suture shuttle 100 is utilized. All aspects of this overall configuration between the mating of ridge 222 and shuttle 100 allow for the location of shuttle 100 with respect to handle 200 to be generally maintained absent purposeful manipulation by the surgeon. That is, the configuration of ridge 222 and the frictional aspects of the devices tend to prevent movement of shuttle 100 along passageway 230. The surgeon is therefore given greater control in utilizing shuttle 100 exactly as dictated by a particular procedure without the need to concentrate on holding the position of shuttle 100 throughout the procedure.

The ridge 222 may have a depression 224 that is aligned with the internal passageway. This depression 224 provides a reference point and guide for the surgeon's finger and suture shuttle 100 while the ridge 222 provides overall support to the elongate body 140 as it spans the contoured portion 220. This support ensures that the suture shuttle 100 does not bend during advancement of the suture shuttle 100 by the surgeon. Both the ridge 222 and depression 224 are ergonomically designed to provide optimum comfort and ease of use during tedious procedures.

One advantage of the configuration of suture passer handle 200 is that it does not necessarily rotationally constrain the suture shuttle 100 when it is loaded into the suture passer handle 200, aside from normal frictional forces. Thus, the surgeon may optionally use his or her thumb to rotate the suture shuttle 100 with respect to passageway 230 while it is loaded into the suture passer handle. This rotational ability is further aided by the depression 224 by providing a resistive surface to roll the suture shuttle 100 while simultaneously preventing bending of the elongate body 110. Additionally, the rounded feature of the elongate body 140, as has been previously described, allows for smooth rotation, thereby limiting the work a surgeon's wrist must perform, and providing the surgeon added flexibility in retrieving a suture. Additionally, the rounded feature allows for smooth tracking during axial translation along the contoured portion 220. Because the suture passer handle allows for tactile manipulation of the suture shuttle by the surgeon, resistive and dynamic feedback to the surgeon is not hindered by a mechanical structure, thereby improving operational feedback.

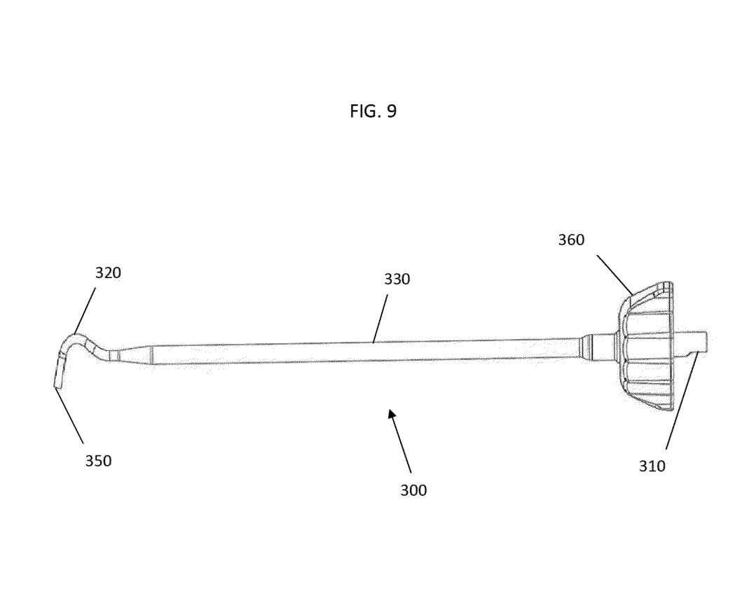

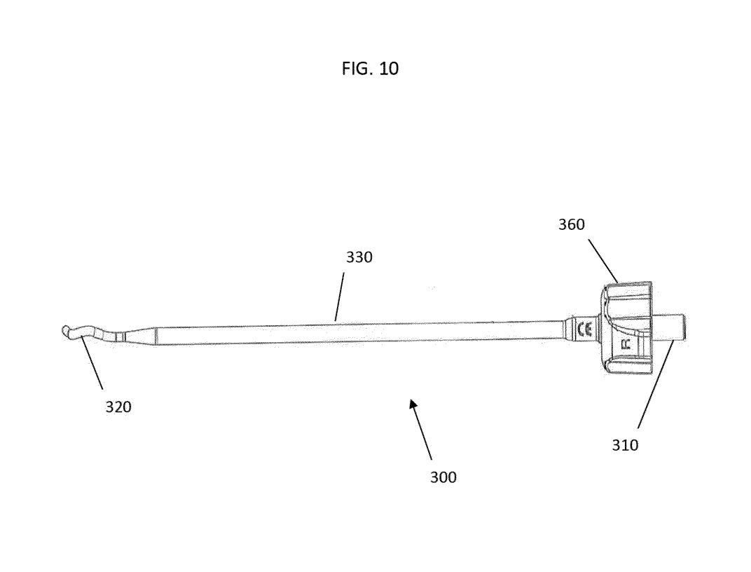

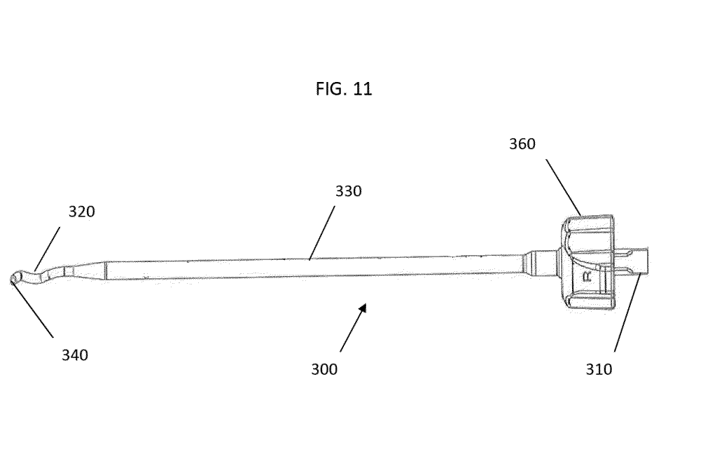

FIGS. 9-11 show a suture passer needle in accordance with an embodiment of the present invention. The suture passer needle 300 generally includes a mating portion 310, a locking mechanism 360, a needle body 330 defining a cannulated passage 340, a needle head 320, and a needle tip 350.

As shown in FIGS. 9-11 and 13, the mating portion 310 of the suture passer needle 300 is dimensioned to fit within the mating portion 240 of the suture passing handle 200, as illustrated in FIGS. 4-8. Further, the mating portion 240 of the suture passer handle 200 is dimensioned to fit within the locking mechanism 360. Rotation of the locking mechanism 360 engages grooves located on the mating portion 240 of the suture passer handle 200 causing the suture passer handle 200 and suture passer needle 300 to lock with respect to each other. Locking the suture passer handle 200 with the suture passer needle 300 brings the internal passageway 230 into fluid communication with the cannulated passage 340 such that the elongate body 140 of the suture shuttle 100 may be passed through the suture passer needle 300 and suture passer handle 200 simultaneously.

FIGS. 9-11 and 13 illustrate an exemplary embodiment of the suture passer needle 300 having a locking mechanism 360 and mating portion 310. Certain additional embodiments may provide a suture passer needle 300 integrated with a suture passer handle 200, thereby precluding the need for the locking mechanism 360 and mating portion 310. Further, other embodiments may provide an entirely different mechanism for mating the suture passer handle 200 with the suture passer needle 300, as would be recognized by a person having ordinary skill in the art.

Referring to FIGS. 1A-3 and 9-11, the cannulated passageway 340 extends through the entire suture passer needle 300. Further, the internal dimensions of the cannulated passageway 340 are dimensionally configured and optimized to receive suture shuttle 100 such that suture shuttle 100 is held stationary until the surgeon tactually interfaces with the elongate body 140. In other words, the cannulated passageway 340 takes advantage of frictional forces between it and suture shuttle 100 to prevent movement of the suture shuttle 100 until the surgeon deliberately moves the suture shuttle 100 through direct, controlled interaction. This is preferably achieved by having the dimension of the inner diameter of the cannulated passageway 340 be substantially the same as the dimension of the outer diameter of body 140 of suture shuttle 100. The outer diameter of body 140 can be defined by the imaginary cylinder coincident with the outermost surfaces of body 140.

Additionally, the suture passer needle has been configured to resist bending during repeated use.

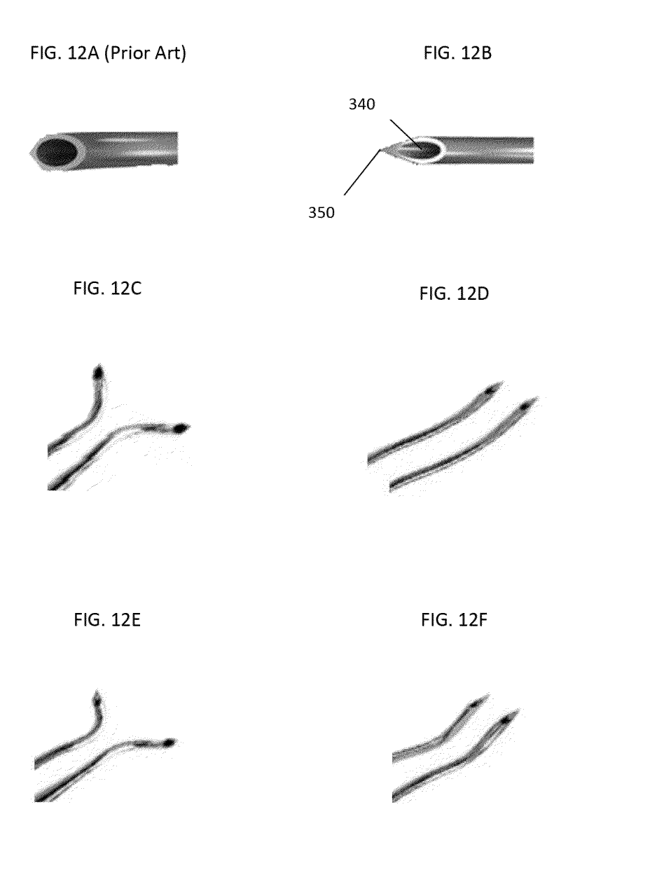

This configuration along with the optimization of the internal dimensions of the suture passer needle 300 has led to an approximately twenty-five percent reduction in the external diameter of the suture passer needle 300 from that of certain existing instruments, thereby reducing the trauma introduced to tissue by the suture passer needle 300. The reduction in diameter from that of certain existing instruments is illustrated by comparing FIG. 12A, which demonstrates certain existing instruments, and FIG. 12B, which shows one embodiment of the suture passer needle 300. Another result of the resistive bending configuration and optimized dimensioning is that, unlike its one-time-use predecessors, the suture passer needle is reposable, meaning that it can be used more than once as desired, but is disposable in nature.

Further illustrated by FIG. 12B, the needle tip 350 may have a sharpened edge for piercing a portion of tissue in order to retrieve and pass a suture through that portion of tissue. As shown in FIGS. 9-11 and 12C-12F, needle head 320 may be provided in various configurations. For example, a kit may be provided for situational adaptability including one or more suture passer needles 300 having needle heads oriented at 45 degrees right and left, crescent shapes of various sizes, 25 degrees right and left and 30 and 60 degrees straight as shown in FIGS. 12C-F, respectively. Further, the various configurations may be color coded, to prevent operation room confusion. However, this is merely an example of the various configurations of a suture passer needle that may be provided. A person of ordinary skill in the art may recognize other variations that would merely be another embodiment of the present invention.

Also included in a kit with one or more suture passer needles can be a reusable suture passer handle, such as handle 200, and one or more suture shuttles, such as shuttle 100. This gives the surgeon flexibility in selecting the most appropriately configured suture passer needle for use in a surgical procedure with a suture passer handle adapted to connect with each of the various needles, with each of the resulting constructs being adapted for use with a suture shuttle, such as shuttle 100.

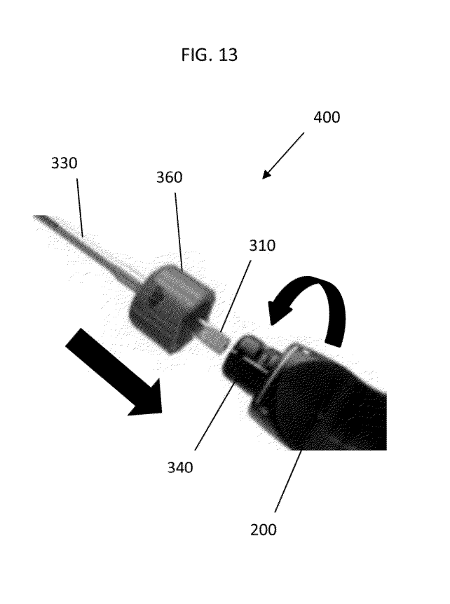

Referring to FIGS. 1A-11 and 13-14B, an example of one method of use will now be described. As shown in FIG. 13, the suture passer handle 200 and suture passer needle 300 are first assembled to form a suture passer assembly 400 by connecting the suture passer needle 300 to the distal end of the suture passer handle 200. In one embodiment, this is achieved by inserting the mating portion 310 of the suture passer needle 300 into the mating portion 240 of the suture passer handle 200, and then rotating locking mechanism 260 in a clockwise direction until suture passer needle is locked to the suture passer handle. This brings internal passageway 230 into fluid communication with the cannulated passageway 340. Other embodiments may omit this step where the suture passer needle 300 is integrated either monolithically or otherwise into the suture passer handle 200.

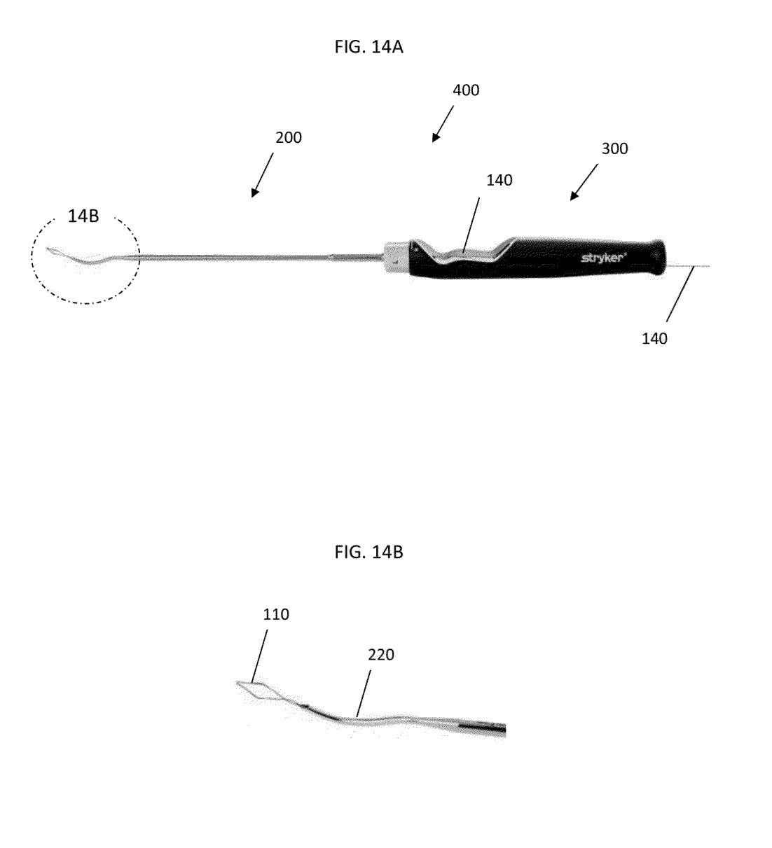

Following the assembly of the suture passer handle 200 and suture passer needle 300, the suture shuttle 100 is loaded into the construct. The distal end of the suture shuttle 100, i.e. the head portion 110, can be slid through the internal passageway 230 at the proximal end of the suture passer handle 200 until head portion 110 resides within the suture passer needle 300. Alternatively, the proximal end of the shuttle 100 can be slid into the cannulated passage 340 at the distal end of suture passer needle 300 until head portion 110 resides within the suture passer needle 300. In either of these methods, care must be taken to move the initially inserted end of the shuttle 100 across the recess 226 so that the shuttle 100 is disposed within both the proximal and distal ends of internal passageway 230, as shown in FIG. 14A. Of course, shuttle 100 could also be loaded into the construct through the interfaces of internal passageway 230 via recess 226.

The surgeon then pierces tissue with needle tip 350 at the intended location and manipulates needle tip 350 into a position at which the suture shuttle 100 can be passed from the device. The suture shuttle 100 is then manipulated to extend out of cannulated passageway 340 away from needle tip 350. Doing this, the surgeon can use a thumb or finger to interact with shuttle 100 within recess 226 to move shuttle 100 either proximally or distally, or to rotate shuttle 100 with respect to cannulated passageway 340 as desired. The surgeon may then further manipulate the suture passer handle 200 or the suture shuttle 100 by further translating or rotating the suture shuttle 100, or any combination thereof, until cavity 120 is accessible to the surgeon for placing a suture therethrough. Such access can be either within the body or outside of the body, such as through a secondary cannula. The manipulation of shuttle 100 to provide access for placing a suture therethrough can be aided by the surgeon grasping shuttle 100, either by hand or with a grasping instrument such as forceps, to place head portion 110 in an accessible location. A suture is then passed through cavity 120, at which point the surgeon then retrieves the suture by retracting head portion 110 into needle head 320, thereby trapping the suture within cannulated passageway 340. This trapping is additionally facilitated by the diamond shape and collapsibility of the head portion 100 by promoting movement of the suture to the narrowest part of head portion 110 and/or pinching it with the flat inner surface 130 of sidewall 150 as head portion 110 is collapsed. The flatness of inner surface 130 provides additional surface area for increased gripping strength.

With the suture disposed through head portion 110 and within cannulated passageway 340, the trapped suture is then passed back through the pierced hole in the tissue by the surgeon retracting suture passer needle 300 out of the pierced tissue through manipulation of suture passer handle 200. At this point in the procedure, the surgeon can then remove the suture from the body, or can pass the suture back through the tissue at a different location by guiding needle tip 350 as described above.

Manipulation of the suture shuttle 100 can occur in several ways. One form of manipulation occurs by translating the suture shuttle 100 in extension or retraction. This can be performed by pressing the elongate body 140 against the depression 224 with a finger and then moving the finger in a distal or proximal direction, thereby translating the suture shuttle 100. This is particularly effective for fine, controlled adjustments. Translation can also be performed by pinching a portion of elongate body 140 that extends proximally from the proximal end of the suture passer handle 300, as shown in FIG. 14A, and then pushing or pulling the elongate body 140, thereby translating the suture shuttle proximally or distally, respectively. This pinching and pushing or pulling adjustment is an advantage to having a long elongate body 140 with respect to the head portion 110 and an internal passageway 230 that extends all the way through the suture passer handle 200. It also is particularly effective when larger movements are desired.

Another form of manipulation is rotation of suture shuttle 100. This may be performed by pressing the elongate body 140 against the ridge 226, or more specifically within the depression 224 if provide to the ridge 226, with a finger and then moving the finger in a lateral direction, thereby rotating the suture shuttle 100. Rotation may also be performed by pinching a portion of the elongate body 140 that extends proximally from the proximal end of the suture passer handle 200 and then rolling the elongate body 140 between the fingers, thereby rotating the suture shuttle 100.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-6. (canceled)

7. A handle connectable to a cannulated needle for forming a suture retrieving system comprising:
a body having a distal end, a proximal end, an internal passageway extending from the distal end to the proximal end, and a recess in the body exposing the internal passageway, the internal passageway being entirely enclosed within the body at least at the proximal end of the body.

8. The handle of claim 7, wherein the recess defines a ridge having an outer boundary that one of intersects or abuts an imaginary cylinder defined by the internal passageway.

9. The handle of claim 8, wherein the ridge includes a depression that is aligned with the internal passageway.

10. The handle of claim 7, wherein an internal diameter of the internal passageway is substantially the same as an outer dimension of a suture shuttle configured to be disposed therein.

11. The handle of claim 7, wherein the body is monolithically constructed and includes no moving parts attached thereto between the distal end and proximal end.

12. The handle of claim 7, further comprising a mating portion disposed at the distal end and having at least one groove configured to lockingly engage a complementary locking mechanism disposed at a proximal end of the cannulated needle.

13. A suture retrieving system comprising:
a handle including a monolithic body having an internal passageway, the body having no moving parts connected thereto between a proximal end and a distal end thereof; and
a needle configured to connect to the distal end of the body and having a cannulated passageway,
wherein the internal passageway is in communication with the cannulated passageway when the needle is connected to the body.

14. The suture retrieving system of claim 13, wherein the internal passageway extends entirely through a length of the body from the proximal end to the distal end.

15. The suture retrieving system of claim 14, wherein the internal passageway is entirely enclosed within the body at least at the proximal end of the body.

16. The suture retrieving system of claim 13, wherein the handle further includes a recess that at least partially intersects the internal passageway and defines a ridge.

17. The suture retrieving system of claim 16, wherein the ridge includes a boundary that one of overlaps or abuts a boundary of an imaginary cylinder defined by the internal passageway.

18. The suture retrieving system of claim 17, wherein the ridge includes a depression that is aligned with the internal passageway.

19. The suture retrieving system of claim 13, wherein the internal passageway has an inner diameter substantially equal to an outer dimension of a suture shuttle configured to be disposed therein.

20. The suture retrieving system of claim 13, wherein the needle is reposable.

21. A suture retrieving device comprising:
a handle including a monolithic body having no moving parts;
a needle connected to a distal end of the handle; and
a passageway extending through the entirety of the needle and handle.

22. The suture retrieving device of claim 21, wherein the passageway is entirely enclosed within the body at least at a proximal end of the handle.

23. The suture retrieving device of claim 21, wherein the handle further includes a recess that at least partially intersects the passageway and defines a ridge.

24. The suture retrieving device of claim 23, wherein the ridge includes a boundary that one of overlaps or abuts a boundary of an imaginary cylinder defined by the passageway.

25. The suture retrieving device of claim 24, wherein the ridge includes a depression that is aligned with the passageway.

26. The suture retrieving device of claim 21, wherein the passageway has an inner diameter substantially equal to an outer dimension of a suture shuttle configured to be disposed therein.
